(19) United States
(12) Patent Application Publication
(10) Pub. No.: US 2007/0060779 A1
(43) Pub. Date: Mar. 15, 2007

(54) PROCESS FOR THE ISOMERIZATION OF XYLENES AND CATALYST THEREFOR

(76) Inventors: Paula L. Bogdan, Mount Prospect, IL (US); Patrick C. Whitchurch, Bossier City, LA (US); Robert B. Larson, Chicago, IL (US); James E. Rekoske, London (GB); Dimitri A. Trufanov, Arlington Heights, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/466,163

(22) Filed: Aug. 22, 2006

Related U.S. Application Data
(60) Provisional application No. 60/717,041, filed on Sep. 14, 2005.

(51) Int. Cl. C07C 5/22

(52) U.S. Cl. 585/482

(57) ABSTRACT

Catalysts of certain combinations of platinum, tin, acidic molecular sieve and aluminum phosphate binder achieve the isomerization and dealkylation activities characteristic of platinum-containing catalysts yet enjoy the low net C₆ naphthenes make properties.

### CROSS REFERENCE TO RELATED APPLICATION

This application claims priority from Provisional Application Ser. No. 60/717,041 filed Sep. 14, 2005, the contents of which are hereby incorporated by reference in its entirety.

### FIELD OF THE INVENTION

This invention pertains to processes for the isomerization of non-equilibrium xylenes and dealkylation of ethylbenzene; to catalysts comprising molecular sieve, platinum and tin in certain relationships with each other and with the molecular sieve and aluminum phosphate binder; and to processes for preferentially depositing platinum on molecular sieve on supports comprising molecular sieve and amorphous aluminum-containing binder.

### BACKGROUND OF THE INVENTION

Catalysts containing platinum and tin have been proposed for use in many chemical and petrochemical reactions including dehydrogenation, dehydrocyclization, aromatization, reforming and isomerization of aliphatics and aromatics. For many of the proposed catalysts, the presence of molecular sieve, acidic or non-acidic, is suggested.

One of the more demanding chemical processes is the isomerization of a non-equilibrium mixture of xylenes and the dealkylation of ethylbenzene. The isomerization processes are practiced on a large, commercial scale to produce para-xylene and, in some instances, ortho-xylene, which have significant uses as raw materials for other chemical processes. For instance, para-xylene is in high demand since it is a raw material to make terephthalic acid for the manufacture of polyester.

The sought xylene isomers, para-xylene and ortho-xylene are often found in a mixture containing meta-xylene which is the most thermodynamically favored of the xylene isomers and with ethylbenzene, another C₈ aromatic isomer. The sought isomers are removed and the remaining isomers are subjected to isomerization to convert a part of the undesired isomer to a sought isomer. For instance, where para-xylene is sought, the para-xylene can be removed by selective crystallization or selective sorption; and ortho-xylene can be recovered by distillation. The remaining xylenes are subjected to isomerization to convert a portion to desired isomers. The isomerization, however, is limited by the equilibria among the isomers. Hence, the isomerate will at best contain about 24 mass-% of para-xylene and about 23 mass-% of the ortho-isomer with the balance being the meta-isomer, based on total xylenes.

The isomerate is recycled for recovery of the sought xylene isomer. The objective in a commercial facility is to ultimately by isomerization and recycle for selective recovery, to convert as much of the xylene feed as possible to the desired isomer and recover that isomer. Complicating the process is the typical presence of another C₈ aromatic, ethylbenzene, in feeds to a xylene recovery operation. To maintain a steady state operation in the cyclic xylene isomer recovery-isomerization loop, ethylbenzene must be removed. Additionally, greater concentrations of ethylbenzene in the recovery—isomerization loop adversely affect the economics of the facility as more energy will be required for the various unit operation. For purposes of illustration of energy requirements reference can be made to a prior art aromatics complex flow scheme disclosed by Meyers in part 2 of the HANDBOOK OF PETROLEUM REFINING PROCESSES, 2d. Edition, in 1997 published by McGraw-Hill. In this process, feed is introduced into a xylene column which separates C₈ aromatics as overhead and heavies are withdrawn from the bottoms. The C₈ aromatics are subjected to a separation process to selectively remove the sought isomer or isomers and then isomerized. Lights are removed from the isomerate by distillation to provide a recycle stream containing C₈ aromatics which is directed to the xylene column.

Removal of ethylbenzene by distillation is problematic due to similarity of boiling points. Accordingly, the most efficient mechanism for its removal is by either ethylbenzene isomerization in which some of the ethylbenzene is converted to xylene in the presence of naphthenes or by dealkylation to yield benzene and ethylene that can be more readily removed from xylenes by distillation.

Accordingly, isomerization processes have been developed that not only isomerize xylenes but also dealkylate ethylbenzene. These processes must effect very distinct and different chemical reactions. First, the xylene isomerization must redistribute the methyl groups on the benzene ring of the xylene isomers. Second, the ethylbenzene must be dealkylated to yield benzene and ethylene, and then third, ethylene must be hydrogenated to ethane. Ideally, these reactions would proceed selectively; however, in practice, numerous side reactions occur. For instance, ethylene could react with a xylene molecule to make methylethylbenzene. Similarly, during the redistribution of methyls on a xylene isomer could lead to the formation of trimethylbenzene and toluene. These and other highers are removed from the recovery—isomerization loop and represent lost xylene. Toluene represents another loss of xylene. Also, the hydrogenation can result in loss of aromatics to naphthenes and acyclic paraffins.

Naphthenes and acyclic paraffins can contaminate products as well as side products that can find some commercial use. One of the more sought side products is benzene. However, stringent specifications need to be met for the benzene to be marketable for certain uses. One such specification is that the benzene purity be at least about 99.85 percent. Naphthenes and paraffins having 6 and 7 carbon atoms (benzene co-boilers) tend to have boiling points close to that of benzene making purification of the benzene by distillation difficult. Accordingly, isomerization processes that generate very low amounts of benzene co-boilers are especially desirable.

Accomplishing the isomerization and dealkylation with a single catalyst while minimizing the undesirable side reactions has proven to be difficult especially since a catalyst needs to perform in a plant environment with adequate catalytic activities and acceptable life. Due to the disparate functions that must be accomplished for isomerization and ethylbenzene dealkylation, proposals have been made to conduct each reaction in a separate zone using different catalysts. This approach, however, increases capital costs and complexities of operation.

U.S. Pat. No. 3,856,872 discloses xylene isomerization and ethylbenzene conversion with a catalyst containing ZSM-5, -12, or -21 zeolite. U.S. Pat. No. 4,362,653 discloses a hydrocarbon conversion catalyst which could be used in the isomerization of isomerizable alkylaromatics that comprises silicalite (having an MFI-type structure) and a silica polymorph. The catalyst may contain optional ingredients. One of the applications of the catalyst is for aromatics isomerization.

U.S. Pat. No. 4,485,185 discloses a catalyst comprising a crystalline aluminosilicate such as MFI and at least two metals which are (a) platinum and (b) at least one other metal from the group consisting of titanium, chromium, zinc, gallium, germanium, strontium, yttrium, zirconium, molybdenum, palladium, tin, barium, cerium, tungsten, osmium, lead, cadmium, mercury, indium, lanthanum and beryllium. The catalyst is said to be useful for the isomerization of aromatic hydrocarbons and reforming of naphtha. The patentees state at column 4, lines 20 to 23 that “Titanium, tin, barium, indium and lanthanum are preferred as metal (b) because they have the great ability to inhibit side reactions. Titanium and tin are most preferred.”

U.S. Pat. No. 4,899,012 discloses the use of a catalyst containing lead, a Group VIII metal, a pentasil zeolite and an inorganic-oxide binder to isomerize xylenes and dealkylate ethylbenzene.

One type of catalyst that has had commercial application for xylene isomerization and ethylbenzene dealkylation comprises platinum on MFI in an inorganic matrix. This type of catalyst generally exhibits a good balance between the desired activities, i.e., approach to xylene isomer equilibrium and ethylbenzene conversion, but, as indicated above, suffers from C₈ aromatic loss through transalkylation and ring saturation.

U.S. Pat. No. 6,143,941 discloses that the use of an amorphous aluminum phosphate binder in a platinum group metal and molecular sieve-containing catalyst in a xylene isomerization and ethylbenzene dealkylation process can substantially reduce xylene loss. The preferred catalyst compositions comprise platinum and MFI with the aluminum phosphate binder. The patentees state: “It is within the scope of the present invention that the catalyst may contain other metal components known to modify the effect of the platinum-group metal component. Such metal modifiers may include without so limiting the invention rhenium, tin, germanium, lead, cobalt, nickel, indium, gallium, zinc, and mixtures thereof. Catalytically effective amounts of such metal modifiers may be incorporated into the catalyst by any means known in the art to effect a homogeneous or stratified distribution.” The patentees in several of the examples deposit platinum or palladium on an aluminum phosphate and MFI molecular sieve support using the tetraamineplatinum chloride or tetraaminepalladium chloride, but no example discloses the use of a metal modifier.

Although the aluminum phosphate binder does reduce xylene loss, these platinum-containing catalysts still leave room for improvement. In copending application Ser. No. 11/226,036, filed Sep. 14, 2005, the applicants disclose that the substitution of molybdenum for platinum in combination with an aluminum phosphate binder and molecular sieve such as MFI unexpectedly reduces xylene loss to even lower levels and in preferred embodiments, the net naphthene make is less than 0.02 mass-% based on total xylenes and ethylbenzene in the feed to the isomerization.

Copending application Ser. No. 11/226,037, filed on Sep. 14, 2005, discloses that the addition of a minor amount of platinum group metal to an isomerization catalyst using molybdenum as the hydrogenation metal component can enhance the approach to isomerization while still retaining a reduced xylene ring loss, especially low naphthene make, as compared to a catalyst containing platinum as the hydrogenation component.

Although platinum has desirable catalytic properties for achieving a close approach to xylene equilibrium during isomerization, it is not evident how to achieve the low levels of xylene loss, especially the low levels of net naphthene make, achievable with other hydrogenation metal components. And it is further not evident how to achieve such low levels of xylene loss, especially low levels of net naphthene make, without adversely affecting other catalyst properties such as activity for ethylbenzene conversion and approach to xylene isomer equilibrium.

Many metals including tin have been proposed as a modifier for platinum-containing catalysts for xylene isomerization and for other chemical reactions. The efficacy of any of these modifiers to achieve, e.g., a low level of net naphthene make without adversely affecting other catalytic properties, is not specifically disclosed in the above prior art.

Tin can have a complex relationship with platinum. For instance, U.S. Pat. No. 6,600,082 discusses platinum and tin-containing dehydrogenation catalysts. By way of background, the patentees observe that “catalysts based on PtSn contain different forms of tin.” They refer to Mössbauer spectroscopy which appears to confirm the existence in a reduced catalyst of an Sn⁰ species in a PtₓSn_{y} type phase (x and y from 1 to 4) in which the tin is in oxidation state 0. They also point to the belief that on alumina, the formation of metallic tin in the reduced state is responsible for the loss in performance of PtSn catalysts. They further add: “A number of documents describe the use of catalysts containing a PtSn phase dispersed on alumina or tin that is essentially in a higher oxidation state than that of metallic tin (U.S. Pat. No. 3,846,283 and U.S. Pat. No. 3,847,794). Under such conditions, the conventional preparation methods used cannot guarantee a close association between tin and platinum, an intimate association between those metals in the catalyst in the reduced state being generally desirable, however, to best exploit the bimetallic effect in processes for transforming organic compounds.” (col. 3, lines 21 to 30)

The background discussion in this patent pertains to alumina supported catalysts for dehydrogenation. One can envisage even further complexities with respect to a catalyst that needs to effect both xylene isomerization and ethylbenzene conversion which contains catalytically-active molecular sieve.

### DEFINITIONS

The following description of the invention, particularly the catalysts of the invention, is made with reference to various test procedures and analyses. The interrelation of the elements of the catalysts of this invention means that a change in one of the components will likely require a change in one or more other components. Additionally, process conditions and the form of the raw materials to make the catalysts of the invention can result in physical differences in the catalyst that may require alterations in ratios of components used. Once understanding the principles of the invention as taught below, one of ordinary skill in the art can readily make and use the invention with reference to Up-Take Analyses.

As used herein, an Up-Take Analysis is performed by immersing known quantities of a sample of the aluminum phosphate used for the binder of the catalyst and a sample of the molecular sieve used in the catalyst in the impregnating solution to be used to make the catalyst. If platinum and tin are co-impregnated in making the catalyst, then the solution will contain both the platinum and tin. If platinum is impregnated after the tin in the process for making the catalyst, then the molecular sieve used for the Up-Take Analysis will contain the intended amount of tin. The immersion is at 25° C. for 1 hour. The immersed samples are withdrawn taking care to remove excess liquid and washed with deionized water. Each of the withdrawn samples is dried at room temperature and then subjected to ICP elemental analysis to determine the amount of platinum in each.

Evaluation Conditions comprise using feed stream containing 15 mass-% ethylbenzene, 25 mass-% ortho-xylene and 60 mass-% meta-xylene; a hydrogen to hydrocarbon ratio of 4:1; a pressure of 1000 kPa gauge; a weight hourly space velocity of 15 hr⁻¹ based upon the mass of the molecular sieve, and a temperature sufficient to convert 75 mass-% of the ethylbenzene with the data taken at 50 hours of operation. These specified conditions are for the purpose of providing common conditions for catalyst evaluation and are not limiting as to the xylene isomerization conditions that may be used in the processes of this invention.

Isomerization Activity is equal to the mass-% of para-xylene to total xylenes in the product obtained under Evaluation Conditions. The relative concentrations of xylenes is determined by gas chromatography using a J&W DB Wax #200-0370 column (60 meters by 0.25 millimeters with 0.5 micron film thickness) available from Agilent Technologies, Inc., Palo Alto, Calif.

Net C₆ Naphthenes Make is the mass-% of C₆ naphthenes in the product obtained under Evaluation Conditions determined by gas chromatography using a J&W PONA column #190915-001 (50 meters by 0.2 millimeter with 0.5 micron film thickness) available from Agilent Technologies.

### SUMMARY OF THE INVENTION

By this invention, novel platinum-containing catalysts are provided, processes for depositing platinum on amorphous aluminum-containing supports are provided, and processes are provided for using platinum and tin-containing catalysts for the isomerization of xylenes and the dealkylation of ethylbenzene that exhibit excellent isomerization activities as well as ethylbenzene dealkylation activities comparable with attenuated aromatic ring hydrogenation activities. Advantageously, the catalysts of this invention can achieve the isomerization and dealkylation activities characteristic of platinum-containing catalysts yet enjoy low net naphthene make.

The catalysts of this invention require certain combinations of platinum, tin, molecular sieve and binder. In one broad aspect the catalysts of this invention comprise: (a) catalytically-effective amount of acidic molecular sieve having a pore diameter of from about 4 to 8 angstroms and a silica to alumina ratio of at least about 20:1, preferably at least about 35:1 and sometimes at least about 40:1; (b) platinum (calculated as atomic platinum) in an amount of between about 150 and 600, preferably between about 150 and 450, parts per million by mass (mass-ppm) based upon the mass of the molecular sieve; (c) amorphous aluminum phosphate binder in an amount of between 1 and 100, preferably 5 to 70, mass parts per 100 mass parts of molecular sieve; and (d) tin wherein the amount of tin (calculated as atomic tin) is in an atomic ratio to platinum in the catalyst of between about 1.2:1 to 30:1, preferably 1.5:1 to 25:1, wherein an Up-Take Analysis provides at least about 90, preferably at least about 95, percent of the platinum on an atomic basis being on a sample of the molecular sieve based upon the total platinum on the sample of the molecular sieve and a sample of the aluminum phosphate.

In another broad aspect, the catalysts of this invention suitable for isomerization of xylenes and conversion of ethylbenzene comprise: (a) a catalytically-effective amount of acidic molecular sieve having a pore diameter of from about 4 to 8 angstroms and a silica to alumina ratio of at least about 20:1, preferably at least about 35:1; (b) a catalytically-effective amount of platinum hydrogenation component on the molecular sieve; and (c) amorphous aluminum phosphate binder and tin both present in an amount sufficient to provide a Net C₆ Naphthenes Make under Evaluation Conditions of less than 0.05, preferably less than about 0.02, mass-% of the total C₈ aromatics in the feed wherein the catalyst under Evaluation Conditions exhibits an Isomerization Activity of at least about 23.0, preferably at least about 23.4, and most preferably at least about 23.6, wherein an Up-Take Analysis provides at least about 90, preferably at least about 95, percent of the platinum on an atomic basis being on a sample of the molecular sieve based upon the total platinum on the molecular sieve and aluminum phosphate samples.

The broad aspects of the processes of this invention comprise contacting a feed stream containing a non-equilibrium admixture of at least one xylene isomer and ethylbenzene wherein preferably between about 1 and 60, and more frequently between about 5 and 35, mass-% of the feed stream is ethylbenzene, with a catalyst comprising (a) a catalytically-effective amount of acidic molecular sieve having a pore diameter of from about 4 to 8 angstroms and a silica to alumina ratio of at least about 20:1, preferably at least about 35:1; (b) a catalytically-effective amount, preferably in an amount of between about 150 and 600, preferably between about 150 and 450, parts per million by mass (mass-ppm) based upon the mass of the molecular sieve, of platinum hydrogenation component on the molecular sieve; (c) amorphous aluminum phosphate binder, preferably in an amount of between 1 and 100, preferably 5 to 70, mass parts per 100 mass parts of molecular sieve, and (d) tin, preferably the amount of tin (calculated as atomic tin) is in an atomic ratio to platinum in the catalyst of between about 1.2:1 to 30:1, preferably 1.5:1 to 25:1, wherein an Up-Take Analysis provides at least about 90, preferably at least about 95, percent of the platinum on an atomic basis being on a sample of the molecular sieve based upon the total platinum on the molecular sieve and aluminum phosphate samples. The isomerization conditions include the presence of hydrogen in a mole ratio to hydrocarbon of between about 0.5:1 to 6:1, preferably 1:1 to 2:1 to 5:1, wherein an Up-Take Analysis provides at least about 90, preferably at least about 95, percent of the platinum on an atomic basis being on a sample of the molecular sieve based upon the total platinum on the molecular sieve and aluminum phosphate samples. Preferably, the isomerization is conducted under at least partially vapor phase conditions. In the preferred aspects of the processes of this invention, the net C₆ naphthenes make under the conditions of the process is less than about 0.05, preferably less than about 0.02, mass-% based on the xylenes and ethylbenzene in the feed.

A further aspect of the invention pertains to processes for co-impregnating platinum and at least one metal modifier a support comprising a catalytically-effective amount of acidic molecular sieve having a pore diameter of from about 4 to 8 angstroms and a silica to alumina ratio of at least about 20:1 and amorphous aluminum-containing binder such as gamma-alumina and aluminum phosphate, in an amount of between 1 and 100 mass parts per 100 mass parts of molecular sieve with platinum comprising contacting an aqueous solution of a compound having a platinum cation, preferably tetraamineplatinum chloride, and a soluble compound of the at least one metal modifier at a temperature of at least about 70° C., preferably between about 80° C. and 150° C., and for a time sufficient to deposit platinum on the support and evaporate water. The impregnation process preferentially provides the platinum deposited on the molecular sieve as compared to the aluminum-containing binder, and the metal modifier is deposited in association with the platinum to provide the modifying effect. The metal modifier may be one or more of tin, rhenium, germanium, lead, cobalt, nickel, indium, gallium, zinc, uranium, dysprosium, thallium, and molybdenum, most preferably tin. While not wishing to be limited to theory, it is believed that an association of the platinum and at least one metal modifier occurs in the impregnating solution which facilitates the preparation of a modified platinum catalyst.

### DETAILED DESCRIPTION OF THE INVENTION

The Catalyst

The catalysts used in the processes of this invention comprise an acidic molecular sieve having a pore diameter of from about 4 to 8 angstroms, platinum and tin in an amorphous aluminum phosphate binder. Examples of molecular sieves include those having Si:Al₂ ratios greater than about 20:1, and often greater than about 35:1 or 40:1, such as the MFI, MEL, EUO, FER, MFS, MTT, MTW, TON, MOR and FAU types of zeolites. Pentasil zeolites such as MFI, MEL, MTW and TON are preferred, and MFI-type zeolites, such as ZSM-5, silicalite, Borolite C, TS-1, TSZ, ZSM-12, SSZ-25, PSH-3, and ITQ-1 are especially preferred.

The zeolite is combined with binder for convenient formation of catalyst particles. The relative proportion of zeolite in the catalyst may range from about 1 to about 99 mass-%, with about 2 to about 90 mass-% being preferred.

The binder or matrix component comprises an amorphous phosphorous-containing alumina (herein referred to as aluminum phosphate) component. The atomic ratios of aluminum to phosphorus in the aluminum phosphate binder/matrix generally range from about 1:10 to 100:1, and more typically from about 1:5 to 20:1. Preferably the aluminum phosphate has a surface area of up to about 450 m²/gram, and preferably the surface area is up to about 250 m²/g.

The amount of the aluminum phosphate binder is preferably sufficient to reduce the transalkylation activity of the catalyst, e.g., co production of toluene and trimethylbenzene. Advantageously, the catalysts of this invention can be characterized as having under Evaluation Conditions, a net make of toluene and trimethylbenzene of less than about 3, preferably less than about 2, mass-% based on the mass of C₈ aromatics (xylenes and ethylbenzene) in the feed.

The aluminum phosphate may be prepared in any suitable manner. One suitable technique for preparing aluminum phosphate is the oil-drop method of preparing the aluminum phosphate which is described in U.S. Pat. No. 4,629,717. This technique involves the gellation of a hydrosol of alumina which contains a phosphorus compound using the well-known oil-drop method. Generally this technique involves preparing a hydrosol by digesting aluminum in aqueous hydrochloric acid at reflux temperatures of about 80° to 105° C. The mass ratio of aluminum to chloride in the sol often ranges from about 0.7:1 to 1.5:1. A phosphorus compound is added to the sol. Preferred phosphorus compounds are phosphoric acid, phosphorous acid and ammonium phosphate. The relative amount of phosphorus and aluminum expressed in atomic ratios ranges from about 10:1 to 1:100, and often 10:1 to 1:10.

If desired, the molecular sieve can be added to the hydrosol prior to gelling the mixture. One method of gelling involves combining a gelling agent with the mixture and then dispersing the resultant combined mixture into an oil bath or tower which has been heated to elevated temperatures such that gellation occurs with the formation of spheroidal particles. The gelling agents which may be used in this process are hexamethylene tetraamine, urea or mixtures thereof. The gelling agents release ammonia at the elevated temperatures which sets or converts the hydrosol spheres into hydrogel spheres. The spheres are then continuously withdrawn from the oil bath and typically subjected to specific aging and drying treatments in oil and in ammoniacal solution to further improve their physical characteristics. The resulting aged and gelled particles are then washed and dried at a relatively low temperature of about 100° to 150° C. and subjected to a calcination procedure at a temperature of about 450° to 700° C. for a period of about 1 to 20 hours.

The combined mixture preferably is dispersed into the oil bath in the form of droplets from a nozzle, orifice or rotating disk. Alternatively, the particles may be formed by spray-drying of the mixture at a temperature of from about 425° to 760° C. In any event, conditions and equipment should be selected to obtain small spherical particles; the particles preferably should have an average diameter of less than about 5.0 mm, more preferably from about 0.2 to 3 mm, and optimally from about 0.3 to 2 mm.

Alternatively, the catalyst may be an extrudate. The well-known extrusion method initially involves mixing of the molecular sieve with optionally the binder and a suitable peptizing agent to form a homogeneous dough or thick paste having the correct moisture content to allow for the formation of extrudates with acceptable integrity to withstand direct calcination. Extrudability is determined from an analysis of the moisture content of the dough, with moisture content in the range of from about 30 to about 50 mass-% being preferred. The dough is then extruded through a die pierced with multiple holes and the spaghetti-shaped extrudate is cut to form particles in accordance with techniques well known in the art. A multitude of different extrudate shapes is possible, including, but not limited to, cylinders, cloverleaf, dumbbell and symmetrical and asymmetrical polylobates. It is also within the scope of this invention that the extrudates may be further shaped to any desired form, such as spheres, by marumerization or any other means known in the art.

Another alternative is to use a composite structure having a core and an outer layer containing molecular sieve and aluminum phosphate. Often, the thickness of the molecular sieve layer is less than about 250 microns, e.g., 20 to 200, microns. The core may be composed of any suitable support material such as alumina or silica, and is preferably relatively inert towards dealkylation. Advantageously, at least about 90 mass-% of the platinum in the catalyst is contained in the outer layer. The catalyst may be in any suitable configuration including spheres and monolithic structures.

The catalyst may contain other components provided that they do not unduly adversely affect the performance of the finished catalyst. These components are preferably in a minor amount, e.g., less than about 40, and most preferably less than about 15, mass-% based upon the mass of the catalyst. These components include those that have found application in hydrocarbon conversion catalysts such as: (1) refractory inorganic oxides such as alumina, titania, zirconia, chromia, zinc oxide, magnesia, thoria, boria, silica-alumina, silica-magnesia, chromia-alumina, alumina-boria, silica-zirconia, phosphorus-alumina, etc.; (2) ceramics, porcelain, bauxite; (3) silica or silica gel, silicon carbide, clays and silicates including those synthetically prepared and naturally occurring, which may or may not be acid treated, for example, attapulgite clay, diatomaceous earth, fuller's earth, kaolin, kieselguhr, etc.; and (4) combinations of materials from one or more of these groups. Often, no additional binder component need be employed.

The catalyst of the present invention may contain a halogen component. The halogen component may be fluorine, chlorine, bromine or iodine or mixtures thereof, with chlorine being preferred. The halogen component is generally present in a combined state with the inorganic-oxide support. The optional halogen component is preferably well dispersed throughout the catalyst and may comprise from more than 0.2 to about 15 mass-%, calculated on an elemental basis, of the final catalyst. The halogen component may be incorporated in the catalyst composite in any suitable manner, either during the preparation of the inorganic-oxide support or before, while or after other catalytic components are incorporated. Preferably, however, the catalyst contains no added halogen other than that associated with other catalyst components.

If desired, the catalyst composite can be dried and then calcined. Drying is often at a temperature of from about 100° to about 320° C. for a period of from about 2 to about 24 or more hours and, usually, calcining is at a temperature of from 400° to about 650° C. in an air atmosphere for a period of from about 0.1 to about 10 hours until the metallic compounds present are converted substantially to the oxide form. If desired, the optional halogen component may be adjusted by including a halogen or halogen-containing compound in the air atmosphere.

The catalytic composite can optionally be subjected to steaming to tailor its acid activity. The steaming may be effected at any stage of the molecular sieve treatment, but usually is carried out on the composite of molecular sieve and binder prior to incorporation of the platinum. Steaming conditions comprise a water concentration of about 1 to 100 vol-%, pressure of from about 100 kPa to 2 MPa, and temperature of from about 600° to about 1200° C.; the steaming temperature preferably is at least about 650° C., more preferably at least about 750° C., and optionally may be about 775° C. or higher. In some cases, temperatures of about 800° to 850° C. for preferably least about one hour.

Alternatively or in addition to the steaming, the composite may be washed with one or more of a solution of ammonium nitrate, a mineral acid, and/or water. Considering the first alternative, the catalyst may be washed with a solution of about 5 to 30 mass-% ammonium nitrate. When acid washing is employed, a mineral acid such as HCl or HNO₃ is preferred; sufficient acid is added to maintain a pH of from more than 1 to about 6, preferably from about 1.5 to 4. The catalyst is maintained in a bed over which the solution and/or water is circulated for a period of from about 0.5 to 48 hours, and preferably from about 1 to 24 hours. The washing may be done at any stage of the preparation, and two or more stages of washing may be employed.

If the molecular sieve is in a metal salt form, the composite is ion-exchanged with a salt solution containing at least one hydrogen-forming cation such as NH₄ or quaternary ammonium to provide the desired acidity. The hydrogen-forming cation replaces principally alkali-metal cations to provide, after calcination, the hydrogen form of the molecular sieve component. Usually, the ion exchange is conducted prior to providing the platinum and tin components.

Platinum is an essential component of the present catalyst. The platinum component may exist within the final catalyst composite as a compound such as an oxide, sulfide, halide, oxysulfide, etc., or as an elemental metal or in combination with one or more other ingredients of the catalyst composite. It is believed that the best results are obtained when substantially all the platinum component exists in a reduced state. The platinum component is preferentially deposited in the molecular sieve. The concentration of platinum (calculated on an atomic basis) based upon the mass of molecular sieve present falls within a relatively narrow range. With too little platinum, not only will the isomerization activity of the catalyst suffer but also the ethylbenzene dealkylation activity suffers and transalkylation side reactions may become more prominent. If the amount of platinum is too great, net naphthene make increases as does transalkylation. Accordingly, by this invention, the concentration of platinum is typically within the range of 150 and 600, preferably between about 150 and 450, mass-ppm based upon the mass of the molecular sieve.

The catalysts of this invention, and the processes of this invention use catalysts, have the platinum component preferentially in the molecular sieve as compared to the amorphous aluminum phosphate. Determining where the platinum component resides in a finished catalyst is difficult and is subject to uncertainties. Accordingly, the Up-Take Analysis procedure is adopted as an indicator of where platinum would be preferentially deposited. It is not, nor is it intended to be, a measure of the amounts and portions of platinum actually deposited on the molecular sieve and on the aluminum phosphate binder. Hence, the catalysts of this invention may actually have a lesser portion of the platinum in the molecular sieve based upon total molecular sieve and aluminum phosphate than indicated by the Up-Take Analysis. Nevertheless, the Up-Take Analysis, by indicating where the platinum is preferentially deposited, is a viable and useful tool for characterizing the catalysts.

The platinum component may be incorporated into the catalyst composite in any suitable manner that achieves the preferential deposition in the molecular sieve. The platinum may be incorporated before, during or after incorporation of the tin component. One method of preparing the catalyst involves the utilization of a water-soluble, decomposable compound of platinum to impregnate the calcined sieve/binder composite. Alternatively, a platinum compound may be added at the time of compositing the molecular sieve component and binder. Complexes of platinum which may be employed according to the above or other known methods include chloroplatinic acid, ammonium chloroplatinate, bromoplatinic acid, platinum trichloride, platinum tetrachloride hydrate, platinum dichlorocarbonyl dichloride, tetraamineplatinum chloride, dinitrodiaminoplatinum, sodium tetranitroplatinate (II), and the like.

The tin component is provided in a critical amount. With insufficient tin, the low net naphthene make is not achieved, but as the amount of tin is increased, the ethylbenzene dealkylation activity decreases. Moreover, the optimal amount of tin will depend upon the amount of platinum in the catalyst. Often, the amount of tin (calculated as atomic tin) is in an atomic ratio to platinum in the catalyst of between about 1.2:1 to 30:1, preferably 1.5:1 to 25:1, and in some instances from about 1.5:1 to 5:1.

The tin component may be incorporated into the catalyst composite in any suitable manner and may be incorporated before, during or after incorporation of the platinum component. One method of preparing the catalyst involves the utilization of a water-soluble, decomposable compound of tin to impregnate the calcined sieve/binder composite. Alternatively, a tin compound may be added at the time of compositing the molecular sieve component and binder. It is essential that the manner in which the tin is provided to the catalyst does not result in undue loss of acidity of the molecular sieve.

The tin compound and composition of the impregnating solution can have an effect on the desired association of tin with platinum group metal. Tin compounds include halogens, hydroxides, oxides, nitrates, sulfates, sulfites, carbonates, phosphates, phosphites, halogen-containing oxyanion salts such as chlorates, perchlorates, bromates, and the like, as will as hydrocarbyl and carboxylate compounds and complexes, e.g., with amines and quaternary ammonium compounds. Exemplary compounds include, but are not limited to tin dichloride, tin tetrachloride, tin oxide, tin dioxide, chlorostannous acid, tetrabutyl tin, tetraethyl tin, ammonium hexachlorostannate, and tetraethylammonium trichlorostannate.

It is within the scope of the present invention that the catalyst composites may contain other metal components. Such metal modifiers may include rhenium, germanium, lead, cobalt, nickel, indium, gallium, zinc, uranium, dysprosium, thallium, molybdenum and mixtures thereof. Catalytically effective amounts of such metal modifiers may be incorporated into the catalysts by any means known in the art to effect a homogeneous or stratified distribution.

The preferred processes of this invention for making the catalyst comprise depositing platinum on a molecular sieve and binder support from a solution, preferably an aqueous solution, in which the platinum is in a cationic form such as tetraamineplatinum chloride. The solution containing the sought amount of platinum, and optionally tin component, and support are combined an mixed and the solvent is evaporated while mixing, preferably at a temperature of at least about 70° C., and more preferably between about 80° C. and 140° C., and the catalyst is dried, e.g., at a temperature of between about 100° C. and 250° C.

The catalysts of this invention are preferably calcined, e.g., at a temperature within the range of about 400° C. and 800° C., preferably in the presence of steam, e.g., about 0.5 to 20 volume percent of the vapor phase, for about 1 to 24, preferably about 1 to 6, hours.

The prepared catalyst, especially due to the calcining, will contain platinum and tin in oxidized states. To obtain the beneficial performance properties, the catalyst is subjected to reducing conditions. Adequate reducing conditions exist for the purposes of activating the catalyst in the isomerization process itself. If desired, the catalyst may be partially or completely pre-reduced. Any suitable reducing technique may be employed. Often the pre-reducing comprises using a gaseous atmosphere comprising at least one of hydrogen and hydrocarbon at elevated temperatures, e.g., from about 250° to 550° C. for 0.5 to 50 hours.

Catalysts may be regenerated. Where the loss of catalytic activity is due to coking of the catalyst, conventional regeneration processes such as high temperature oxidation of the carbonaceous material on the catalyst may be employed.

The Process

The feed stocks to the aromatics isomerization process of this invention comprise non-equilibrium xylene and ethylbenzene. These aromatic compounds are in a non-equilibrium mixture, i.e., at least one C₈ aromatic isomer is present in a concentration that differs substantially from the equilibrium concentration at isomerization conditions. Thus, a non-equilibrium xylene composition exists where one or two of the xylene isomers are in less than equilibrium proportion with respect to the other xylene isomer or isomers. The xylene in less than equilibrium proportion may be any of the para-, meta- and ortho-isomers. As the demand for para- and ortho-xylenes is greater than that for meta-xylene, usually, the feed stocks will contain meta-xylene. Generally the mixture will have an ethylbenzene content of about 1 to about 60 mass-%, an ortho-xylene content of 0 to about 35 mass-%, a meta-xylene content of about 20 to about 95 mass-% and a para-xylene content of 0 to about 30 mass-%. Usually the non-equilibrium mixture is prepared by removal of para-, ortho- and/or meta-xylene from a fresh C₈ aromatic mixture obtained from an aromatics-production process. The feed stocks may contain other components, including, but not limited to naphthenes and acyclic paraffins, as well as higher and lower molecular weight aromatics.

The alkylaromatic hydrocarbons may be used in the present invention as found in appropriate fractions from various refinery petroleum streams, e.g., as individual components or as certain boiling-range fractions obtained by the selective fractionation and distillation of catalytically cracked or reformed hydrocarbons. Concentration of the isomerizable aromatic hydrocarbons is optional; the process of the present invention allows the isomerization of alkylaromatic-containing streams such as catalytic reformate with or without subsequent aromatics extraction to produce specified xylene isomers and particularly to produce para-xylene.

According to the process of the present invention, the feedstock, in the presence of hydrogen, is contacted with the catalyst described above. Contacting may be effected using the catalyst system in a fixed-bed system, a moving-bed system, a fluidized-bed system, and an ebullated-bed system or in a batch-type operation. In view of the danger of attrition loss of valuable catalysts and of the simpler operation, it is preferred to use a fixed-bed system. In this system, the feed mixture is preheated by suitable heating means to the desired reaction temperature, such as by heat exchange with another stream if necessary, and then passed into an isomerization zone containing catalyst. The isomerization zone may be one or more separate reactors with suitable means therebetween to ensure that the desired isomerization temperature is maintained at the entrance to each zone. The reactants may be contacted with the catalyst bed in upward-, downward-, or radial-flow fashion.

The isomerization is conducted under isomerization conditions including isomerization temperatures generally within the range of about 100° to about 550° C. or more, and preferably in the range from about 150° to 500° C. The pressure generally is from about 10 kPa to about 5 MPa absolute, preferably from about 100 kPa to about 3 MPa absolute. The isomerization conditions comprise the presence of hydrogen in a hydrogen to hydrocarbon mole ratio of between about 0.5:1 to 6:1, preferably about 1:1 or 2:1 to 5:1. One of the advantages of the processes of this invention is that relatively low partial pressures of hydrogen are still able to provide the sought selectivity and activity of the isomerization and ethylbenzene conversion. A sufficient mass of catalyst (calculated based upon the content of molecular sieve in the catalyst composite) is contained in the isomerization zone to provide a weight hourly space velocity with respect to the liquid feed stream (those components that are normally liquid at STP) of from about 0.1 to 50 hr⁻¹, and preferably 0.5 to 25 hr⁻¹.

The isomerization conditions may be such that the isomerization is conducted in the liquid, vapor or at least partially vaporous phase. For convenience in hydrogen distribution, the isomerization is preferably conducted in at least partially in the vapor phase. When conducted at least partially in the vaporous phase, the partial pressure of C₈ aromatics in the reaction zone is preferably such that at least about 50 mass-% of the C₈ aromatics would be expected to be in the vapor phase. Often the isomerization is conducted with essentially all the C₈ aromatics being in the vapor phase.

Usually the isomerization conditions are sufficient that at least about 10, preferably between about 20 and 80 or 90, percent of the ethylbenzene in the feed stream is converted. Generally the isomerization conditions do not result in a xylene equilibrium being reached. Often, the mole ratio of xylenes in the product stream is at least about 80, say, between about 85 and 99, percent of equilibrium under the conditions of the isomerization. Where the isomerization process is to generate para-xylene, e.g., from meta-xylene, the feed stream contains less than 5 mass-% para-xylene and the isomerization product comprises a para-xylene to xylenes mole ratio of between about 0.20:1 to 0.25:1 preferably at least about 0.23:1, and most preferably at least about 0.236:1.

The particular scheme employed to recover an isomerized product from the effluent of the reactors of the isomerization zone is not deemed to be critical to the instant invention, and any effective recovery scheme known in the art may be used. Typically, the isomerization product is fractionated to remove light by-products such as alkanes, naphthenes, benzene and toluene, and heavy byproducts to obtain a C₈ isomer product. Heavy byproducts include dimethylethylbenzene and trimethylbenzene. In some instances, certain product species such as ortho-xylene or dimethylethylbenzene may be recovered from the isomerized product by selective fractionation. The product from isomerization of C₈ aromatics usually is processed to selectively recover the para-xylene isomer, optionally by crystallization. Selective adsorption is preferred using crystalline aluminosilicates according to U.S. Pat. No. 3,201,491. Improvements and alternatives within the preferred adsorption recovery process are described in U.S. Pat. No. 3,626,020, U.S. Pat. No. 3,696,107, U.S. Pat. No. 4,039,599, U.S. Pat. No. 4,184,943, U.S. Pat. No. 4,381,419 and U.S. Pat. No. 4,402,832, incorporated herein by reference.

### EXAMPLES

The following examples are presented only to illustrate certain specific embodiments of the invention, and should not be construed to limit the scope of the invention as set forth in the claims. There are many possible other variations, as those of ordinary skill in the art will recognize, within the spirit of the invention.

### Example I

Catalyst samples are prepared.

Catalyst A: Steamed and calcined aluminum-phosphate-bound MFI zeolite spheres are prepared using the method of Example I in U.S. Pat. No. 6,143,941. The pellets are impregnated with an aqueous solution of 1:2:6 moles of tin(II)chloride: ethylenediamminetetraacetic acid: ammonium hydroxide and tetra-ammine platinum chloride to give 0.023 mass-% platinum and 0.20 mass-% tin on the catalyst after drying and calcination in air with 3% steam at 538° C.

Catalyst B: Steamed and calcined aluminum-phosphate-bound MFI zeolite spheres are prepared using the method of Example I in U.S. Pat. No. 6,143,941. The pellets are impregnated with an aqueous solution of 1:2:6 moles of tin(II)chloride:ethylenediamminetetraacetic acid:ammonium hydroxide and tetra-ammine platinum chloride to give 0.039 mass-% platinum and 0.29 mass-% tin on the catalyst after drying and calcination in air with 3% steam at 538° C.

Catalyst C: Steamed and calcined aluminum-phosphate-bound MFI zeolite spheres are prepared using the method of Example I in U.S. Pat. No. 6,143,941. The pellets are impregnated with an aqueous solution of 1:2:6 moles of tin(II)chloride: ethylenediamminetetraacetic acid: ammonium hydroxide and tetra-ammine platinum chloride to give 0.046 mass-% platinum and 0.11 mass-% tin on the catalyst after drying and calcination in air with 3% steam at 538° C.

### Example II

Catalysts A, B and C are evaluated in a pilot plant for the isomerization of a feed stream containing 7 mass-% ethylbenzene, 1 mass-% para-xylene, 22 mass-% ortho-xylene and 70 mole-percent meta-xylene. The pilot plant runs are at a hydrogen to hydrocarbon ratio of 4:1, total pressure of 1200 kPa, and weight hourly space velocity of 10 based on the total amount of catalyst loaded. The pilot plant runs are summarized in the following table. The product data are taken at approximately 50 hours of operation.

| | Catalyst | | |
|---|---|---|---|
| | A | B | C |
| Sn/Pt atomic ratio | 14 | 12 | 4 |
| EB Conversion, % | 75 | 75 | 75 |
| WABT*, ° C. | 385 | 390 | 385 |
| Para-xylene/xylene | 23.8 | 23.8 | 23.8 |
| Toluene + Trimethylbenzene, mass-% yield | 1.8 | 1.6 | 2.0 |
| C₆ Naphthenes, mass-% yield | 0.02 | 0.04 | 0.08 |

*weighted average bed temperature

### Example III

Catalysts are prepared using similar procedures and components as in Example I and are evaluated in a similar manner to that described in Example II. The following table sets forth the catalyst compositions and performance. The benzene purity (BZ purity) is the mass percent benzene based upon total benzene and naphthenes and paraffins of 6 and 7 carbon atoms. The table also sets forth the temperature of the impregnation of each catalyst. The evaluation is at 75 percent conversion of ethylbenzene.

| | | | | | | Para- |
|---|---|---|---|---|---|---|
| | Pt | Sn %- | Impreg | WABT | BZ purity | xylene/ |
| Catalyst | ppm-m | mass | Temp ° C. | ° C. | %-mass | xylene % |
| M | 220 | 0.19 | 130 | 391 | 99.9 | 23.70 |
| N | 280 | 0.22 | 100 | 393 | 99.7 | 23.65 |
| O | 430 | 0.096 | 130 | 388 | 99.6 | 23.80 |
| P | 280 | 0.22 | 130 | 396 | 99.8 | 23.74 |
| Q | 450 | 0.052 | 130 | 388 | 99.6 | 23.83 |
| R | 380 | 0.07 | 130 | 384 | 99.7 | 23.45 |
| S | 230* | 0.07 | 130 | 387 | 99.9 | 23.40 |
| T | 350 | 0.04 | 130 | 387 | 99.7 | 23.60 |
| U | 370 | 0.04 | 130 | 387 | 99.3 | 23.60 |

*Catalyst S, when analyzed appears to not be at target platinum concentration.

1. A process for isomerizing xylenes and dealkylating ethylbenzene contained feed stream comprising a non-equilibrium admixture of at least one xylene isomer and ethylbenzene comprising contacting under isomerization conditions including the presence of hydrogen the feed stream with a catalyst comprising:
(a) a catalytically-effective amount of acidic molecular sieve having a pore diameter of from about 4 to 8 angstroms and a silica to alumina ratio of at least about 20:1;
(b) a catalytically-effective amount of platinum hydrogenation component on the molecular sieve;
(c) amorphous aluminum phosphate binder and
(d) tin,
wherein an Up-Take Analysis provides at least about 90 percent of the platinum on an atomic basis being on a sample of the molecular sieve based upon the total platinum on the molecular sieve and aluminum phosphate samples.

2. The process of claim 1 wherein the naphthene net make is less than about 0.05 mass-% based on the xylenes and ethylbenzene in the feed stream.

3. The process of claim 2 wherein the feed stream contains between about 5 and 35 mass-% ethylbenzene.

4. The process of claim 3 wherein the feed stream contains less than 5 mass-% para-xylene and the isomerization product comprises a para-xylene to xylenes mole ratio of at least about 0.23:1.

5. A process for isomerizing xylenes and dealkylating ethylbenzene containing feed stream comprising a non-equilibrium admixture of at least one xylene isomer and ethylbenzene comprising contacting the feed stream under isomerization conditions including the presence of hydrogen with a catalyst comprising.
(a) catalytically-effective amount of acidic molecular sieve having a pore diameter of from about 4 to 8 angstroms and a silica to alumina ratio of at least about 20:1;
(b) platinum (calculated as atomic platinum) in an amount of between about 150 and 600 mass-ppm based upon the mass of the molecular sieve;
(c) amorphous aluminum phosphate binder in an amount of between 1 and 100 mass parts per 100 mass parts of molecular sieve; and
(d) tin wherein the amount of tin (calculated as atomic tin) is in an atomic ratio to platinum in the catalyst of between about 1.2:1 to 30:1,
wherein an Up-Take Analysis provides at least about 90 percent of the platinum on an atomic basis being on a sample of the molecular sieve based upon the total platinum on the molecular sieve and aluminum phosphate samples.

6. The process of claim 1 wherein the tin and the platinum of the catalyst are provided by an aqueous solution of a compound having a platinum cation and a soluble compound of the at least one metal modifier which is contacted with the molecular sieve at a temperature of at least about 70° C. and for a time sufficient to deposit platinum and the at least one metal modifier on the support and evaporate water.

7. The process of claim 5 wherein the tin and the platinum of the catalyst are provided by an aqueous solution of a compound having a platinum cation and a soluble compound of the at least one metal modifier which is contacted with the molecular sieve at a temperature of at least about 70° C. and for a time sufficient to deposit platinum and the at least one metal modifier on the support and evaporate water.
